(19) United States
(12) Patent Application Publication
(10) Pub. No.: US 2018/0325959 A1
(43) Pub. Date: Nov. 15, 2018

(54) METHODS OF TREATING ERECTILE DYSFUNCTION

(71) Applicant: Creative Medical Technologies, Inc., Phoenix, AZ (US)

(72) Inventors: Thomas ICHIM, San Diego, CA (US); Amit PATEL, Salt Lake City, UT (US); Annette MARLEAU, San Diego, CA (US)

(21) Appl. No.: 15/590,668

(22) Filed: May 9, 2017

(51) Int. Cl. A61K 35/48; A61K 9/00; A61K 35/12; C12N 5/071; C12N 5/10; C12Q 1/68

(52) U.S. Cl. CPC A61K 35/48; A61K 9/0034; A61K 35/12; C12N 2501/10; C12N 5/10; C12Q 1/68; C12N 5/0683

(57) ABSTRACT

Therapies for treating erectile dysfunction using regenerative cells and therapeutic energy treatments are disclosed herein. Said therapeutic energy treatments can be selected from one or more of the following methods: electrical stimulation/electroacupuncture, low-level laser irradiation, and extracorporeal shockwave therapy. The combination treatments described herein are useful for restoring components of the penile anatomy that are associated with erectile dysfunction, in particular, nerves, blood vessels and/or smooth muscle cells.

### FIELD OF THE INVENTION

The invention pertains to the field of erectile dysfunction. Specifically, this invention relates to therapies for treating erectile dysfunction using regenerative cells and therapeutic energy treatments. Said therapeutic energy treatments are selected from one or more of the following: electrical stimulation/electroacupuncture, low-level laser irradiation, and extracorporeal shockwave therapy. The combination treatments embodied by the present invention are useful for restoring components of the penile anatomy that are associated with erectile dysfunction, in particular, nerves, blood vessels and/or smooth muscle cells.

### BACKGROUND OF THE INVENTION

### Erectile Dysfunction: Pathophysiology and Treatments

Erectile dysfunction (ED) is a clinical problem that affects a significant proportion of the male population and increases in incidence with aging. In men ages 40 to 70 years old, the Massachusetts Male Aging Study found the prevalence of erectile dysfunction to be 52% [1]. By the fourth and fifth decades of life, men frequently have diminished erectile function, marked by a reduced ability to maintain an erection during sex and an increased refractory period between erectile events [2]. The causes of erectile dysfunction are varied and are typically divided into two major categories: organic and psychological. ED is also the most prevalent complication of post-radical prostatectomy (prostate cancer treatment), which occurs in up to 70% of these patients even in those treated using nerve sparing techniques [3, 4]. ED can be caused by radiation therapy for prostate cancer, which is associated with arterial damage and decreased motor function in the pudendal nerve [5]. The present invention deals specifically with organic causes of ED, which include arteriogenic, vasculogenic, neurogenic, hormonal and drug- and surgery/radiation-induced causes. There have been reports of strong positive correlations between ED and aging. In addition, positive correlations have been reported between ED and hypertension, coronary heart disease, diabetes, and impaired cognitive function [6-8].

Structurally, the penis is comprised of three erectile bodies, two parallel bodies termed corpora cavernosa, and the corpus spongiosum, a mass of spongy tissue surrounding the male urethra. These three erectile bodies are heavily vascularized and contain large proportion of smooth muscle cells. Erection is caused by neurologically induced relaxation of smooth muscle cells in the erectile bodies, which allows influx and accumulation of blood into the sinusoids. As blood accumulates, the outflow of blood is prevented by pressure from a structure called the tunica albuginea (a fibrous layer surrounding the corpus cavernosum) against the venous plexus. This creates a veno-occlusive mechanism that traps the blood and allows the penis to remain rigid. Thus, while the sinusoids contain relatively little blood when the penis is in a relaxed state, penile erection requires that these sinusoids dilate and become engorged and also that this response can be sustained.

The penis is innervated by the dorsal penile and perineal nerves [9]. These nerves are a continuation of sympathetic and parasympathetic autonomic nerves as well as sensory and motor somatic nerves. The somatic sensory system is responsible for transmitting information about the external environment. A fully functional penile erection requires coordinated changes in output from various levels of the central nervous system and at least three sets of peripheral nerves (thoracolumbar sympathetic, sacral parasympathetic, and pelvic somatic). Physiological erections originate in the central nervous system involving both sympathetic and parasympathetic pathways. The sympathetic component inhibits erections, whereas the parasympathetic system comprises excitatory pathways that mediate arousal. Parasympathetic nervous activation causes relaxation of smooth muscle and dilation of the arteries in the corpora cavernosum and the corpus spongiosum. This dilation, in combination with the veno-occlusive mechanism that traps blood, allows an erection to occur. Parasympathetic activation causes upregulation of nitric oxide (NO) production by nonadrenergic, noncholinergic nerves, as well as endothelium lining the penile arteries and cavernosal sinusoids. NO is believed to be the main vasoactive nonadrenergic, noncholinergic neurotransmitter and chemical mediator of penile erection. Accumulation of NO increases production of cyclic guanosine monophosphate (cGMP) through activation of the enzyme guanylyl cyclase. cGMP acts as a second messenger which leads to decrease calcium uptake into the cavernous and endothelial-lining smooth muscles, thus causing relaxation and erection. While impaired arterial inflow of blood into the penis and/or an impaired veno-occlusive mechanism are often regarded as the most common causes of ED, the incidence of altered neural function may be under-estimated. There is evidence that neuronal-derived NO is essential for initiation and maintenance of an erection [10]. Neural deficits in the penis or in peripheral nerves that ultimately transmit signals to the penis may also co-exist with biological dysfunctions such as a loss of smooth muscle mass or arterial dysfunction. Treatments must therefore address the varied biological mechanisms that precipitate this condition, and ideally, to treat multiple biological mechanisms that may be operating to account for ED.

Since phosphodiesterase (PDE)-5 is involved in the breakdown of cGMP, the inhibition of PDE-5 has been chosen as a pharmacological goal of medications such as Viagra (sildenafil), Cialis (tadalafil) and Cialis (vardenafil). Unfortunately, a substantial number of patients (approximately 30%) are resistant to PDE5 inhibitors, including patients with advanced neurologic damage and vascular diseases [11]. For example, the response rate to sildenafil decreased from 72% in men 18-49 y of age to 53% in men 50 y or older [12]. Sildenafil therapy failed mainly in men with diabetes, non-nerve sparing radical prostatectomy, and high disease severity [13, 14]. One reason for the lack of effectiveness of PDE5 inhibitors may be the loss of erectile tissue. Notably, one line of thought is that aging-related vascular smooth muscle apoptosis is a significant contributor to ED in both young (<40 years of age) and older men [2]. According to this mechanism, smooth muscle loss within the entire vascular tree in the periphery is associated with coincident smooth muscle loss in the penis, leading to the first signs of ED that are marked by symptomatic venous leakage and difficulty maintaining an erection.

Alternatives to oral medications for ED currently include surgical intervention (destructive and irreversible); vacuum devices (moderate efficacy, may be difficult for some men to use and may cause penile trauma); and injections with vasoactive compounds into the corpus cavernosum for the purposes of producing an erection.

Regenerative Cells in the Context of ED

Because of their diverse tissue-healing capabilities, stem cells have attracted much attention as a therapeutic measure for the treatment of several conditions including ED. Mesenchymal stem cells (MSC) are one therapeutically attractive source of stem cells. In addition to “universal donor” properties, MSC have been demonstrated to be capable of differentiating along the orthodox pathways, which includes bone, cartilage, and adipose tissue, as well as along the non-orthodox pathways, including pancreatic, cardiac, neural and hepatic tissues [15]. However, MSCs do not have unlimited proliferative capacity and their ability to differentiate into multiple lineages is influenced by multiple factors including donor age [16] and presumably numerous other variables.

Therefore, while it has been anticipated that MSC could be used for replacement of dysfunctional cells via their capacity to differentiate into tissue cells, the current paradigm is that MSC support resident progenitor cells via paracrine mechanisms including angiogenic, anti-inflammatory and anti-fibrotic mechanisms.

Regenerative cells (stem cells, progenitor cells and the like) including but not limited to MSC, are capable of elaborating their regenerative functions including tissue-specific differentiation in response to tissue injury through a series of molecular events. Tissue injury is associated with the activation of immune/inflammatory cells including macrophages, neutrophils, T cells and B cells, which are recruited by factors from apoptotic cells, necrotic cells, damaged microvasculature and stroma [17, 18]. Inflammatory mediators, such as TNF-α, IL-1β, free radicals, chemokines and leukotrienes, are often produced by phagocytes in response to damaged cells and soluble signals from said cells.²¹ Thus, these inflammatory molecules and immune cells, together with endothelial cells and fibroblasts, orchestrate changes in the microenvironment that result in the mobilization and differentiation of stem cells into stromal and/or replacement of damaged tissue cells. These stem cells can be tissue-resident or be recruited from the bone marrow. It is also known in the art that stem cells can be injected for therapeutic purposes For therapeutic purposes, injected stem cells can emigrate to or be injected directly into a damaged tissue. One major issue in stem cell therapeutics has been the identification of means for improving the delivering of and/or affixing stem cells (or regenerative cells as used herein) to a target tissue.

Electrical Stimulation, Electroacupuncture, and Transcutaneous Electrical Stimulation

In China, the insertion of acupuncture needles into acupuncture points to treat diseases has been practiced for at least 2,000 years. Electro-acupuncture (EA) is a type of therapy in which a needle inserted into an acupoint is attached to a trace pulse current with the purpose of producing synthetic electric and needling stimulation. Acupuncture is an alternative medicine that treats patients by insertion and manipulation of needles in the body at selected points. See, Novak, Patricia D. et al (1995). Dorland's Pocket Medical Dictionary (25th ed.), Philadelphia: (W.B. Saunders Publisher), ISBN 0-7216-5738-9. Indeed, the field of neurostimulation deals with identifying locations in the body where electrical stimulation can be applied in order to provide a therapeutic effect for a patient. Electroacupuncture, which is similar to traditional acupuncture in that the same points along the body are stimulated using needles that are attached to a device that generates electric pulses; however, two needles at a time are utilized such that the current can pass between them. A variation of the EA technique, termed transcutaneous electrical nerve stimulation (TENS), uses electrodes that are taped to the surface of the skin instead of being inserted, thereby avoiding the use of needles. TENS involves attaching electrodes to the outer surface of the tissue without breaking the skin, unlike the percutaneous route whereby electrical stimulation is delivered via an electrode placed under the skin via needle puncture.

This type of therapy has been used for in situ analgesia and tissue repair and regeneration, for example, most common to treat nerve damage in spinal cord injury. In one published study investigating the effects of EA on stem cell therapy in an animal model, the level of cAMP and neurotrophin-3, the numbers of vital and differentiated MSCs, the numbers of regenerated axons in and near the lesion site of the injured spinal cord were all significantly increased in the treatment group that received MSC and EA as compared to control groups that received MSC or EA treatment alone [19].

Techniques for using electrical devices, including external EA devices, for stimulating peripheral nerves and other body locations for treatment of various illnesses are known in the art. U.S. Pat. No. 7,203,548, issued to Whitehurst et al., discloses use of an implantable miniature neurostimulator, referred to as a “microstimulator,” that can be implanted into a desired tissue location and used as a therapy for cavernous nerve stimulation. The microstimulator has a tubular shape, with electrodes at each end. U.S. patent application Ser. No. 13/784,573 describes a less invasive means for treating ED using EA a small, leadless, implantable electroacupuncture device (IEAD) powered by a small disc primary battery. The methods and devices disclosed for treating ED with EA utilize either external or implantable devices for delivering electrical stimulation to the penis.

In the context of the present invention, electrical stimulation is utilized to stimulate regenerative cells in vitro. Said regenerative cells are subsequently administered to an individual in need thereof as a treatment for ED. Methods for electrical stimulation of cells in vitro are known in the art. For example, electric fields within the endogenous physiological range (tens to hundreds of mV/mm) have been utilized to stimulate the differentiation of various cell types, including fibroblasts [20], human mesenchymal stem cells [21], human cardiac progenitor cells (c-kit/Sca-1) [22], and mouse adipose-derived stem cells [23] into immature cardiomyocyte-like cells.

Laser Irradiation

A laser (light amplification by stimulated emission of radiation) is a device that generates electromagnetic radiation that is relatively uniform in wavelength, phase, and polarization [24]. Treatment with low level lasers, termed “photobiomodulation”, has been shown to exert diverse effects on the growth and activity of cellular populations that is dependent upon the intensity of stimulation, allowing for either stimulation or inhibition of cellular functions. Visible, infrared or ultraviolet spectra of light can be utilized in the art. It is known in the art that the visible light spectrum ranging from 600-700 nm is capable of inducing the proliferation and differentiation of numerous cell types including fibroblasts, osteoblasts, endothelial cells, MSC, and adipose-derived stem cells [25-29]. In the art, this is referred to as “low level laser irradiation.” Low-level laser irradiation is used in the art to enhance stem cell yields in culture by increasing proliferation [24]. Low-level laser therapy also comprises a medical therapy wherein human tissue is irradiated with a low-powered laser to induce therapeutic effects. It the art of regenerative medicine, it is known that laser therapy can improve wound healing and exert beneficial effects in reducing pain and inflammation. In the art, the types of laser most frequently used for wound healing and tissue repair are helium-neon (He—Ne) lasers and diode lasers, including gallium-aluminum-arsenic (Ga—Al—As), arsenic-gallium (As—Ga), and indium-gallium-aluminum-phosphide (In—Ga—Al—P) lasers [24].

Low-level laser irradiation has been applied to patients with paroxetine-induced penile anesthesia, i.e. a loss of sensitivity of the penis coincident with scrotum hypesthesia, anejaculation and erectile difficulties [30]. Treatment was performed using single and and multi diode pulsed laser probes for 20 sessions of 15min each. Clinical improvement of glans penis sensitivity was reported, however, this therapy did not improve erectile function.

Mechanistically, low-level laser irradiation has been shown to exert numerous molecular effects within cells. For example, low-level laser irradiation can generate oxygen radicals in exposed cells, and can upregulate nitric oxide and nitric oxide synthase (iNOS), which in turn leads to changes in expression of numerous transcription factors including but not limited to hypoxia inducible factor-1 (HIF-1), NF-kappaB, and activator-protein 1 (AP-1) [24]. Low-level laser irradiation can suppress inflammatory responses by modulating intracellular cyclic AMP levels and NF-kappB activity, thereby also reducing pro-inflammatory cytokine gene expression [31]. The intracellular mechanisms elicited within cells treated with low-level laser irradiation can lead to cell proliferation, differentiation or apoptosis. One factor known in the art to control these divergent cellular fates is the laser fluence, defined as the optical energy delivered per area of tissue in the case of the present invention. For a particular cell type and treatment, fluence is often considered by practitioners in the art as the “dose” of therapy and is measured in J/cm² Mechanistically, increasing the “dose” of low level laser irradiation can lead to the generation of higher levels of reactive oxygen species and can be associated with cellular apoptosis [32]

Extracorporeal Shockwave Therapy (EST)

EST is a non-invasive procedure involving the use of computer-controlled sound waves. This procedure was first medically used for lithotripsy of uroliths and gallstones in humans and animals. Since the 1990s, EST has been commonly used in the art for treating musculoskeletal disorders in veterinary medicine. In numerous indications, there is evidence that low intensity treatment acts by stimulating angiogenesis (sprouting of existing vessels) and local neovascularization (new blood vessel growth). Indications that have been examined include any number of tendon, joint, and muscle conditions (shoulder rotator cuff pain, plantar fasciitis, tendonitis), to promote bone healing, wound healing, angina, chronic pelvic pain syndrome, and in ED.

Experimental evidence has accrued demonstrating that EST stimulates recruitment of MSC to the penis and promotes partial restoration of smooth muscle [33, 34]. Clinical studies support a degree of benefit of EST for erectile dysfunction. Olsen et al. administered five EST treatments to individuals with ED over a period of five weeks and found that 57% (20 men) and 9% (5 men) of the treatment vs. control groups were able to obtain an erection and have sexual intercourse without medication after 5 weeks. 19% (7 men) and 23% (9 men) of the treatment vs. control groups were able to have intercourse without medication after 6 months [35]. Vardi et al. reported a study of men with organic ED that were treated with two sessions of low intensity shock wave therapy per week for three weeks, followed by a 3-week no-treatment interval, and then a second 3-week treatment period of two treatment sessions per week [36] . At the 6-month follow-up visit, 10 men of the 20 treated reported that they still had spontaneous erections that were sufficient for penetration. In another study, Gruenwald et al. treated men with ED with two treatment sessions per week for 3 weeks, which were repeated after a 3-week no-treatment interval. They reported that Of 29 men, 8 men achieved achieved normal erections and 21 were able to achieve vaginal penetration with oral PDE5i therapy [37]. The ability to achieve partial long-term success with this therapy was attributable to improved cavernosal blood flow, increased smooth muscle functions and/or improved penile endothelial function, possibly attributable to mobilization and/or increased activity of endogenous stem cells in these patients. On this basis, methods for improving the regenerative capabilities within this tissue, utilizing cells having sufficient regenerative activity capable of ameliorating and reversing the pathological processes that contribute to ED, are continually sought.

### BRIEF SUMMARY OF THE INVENTION

Embodiments herein are directed to methods of treating erectile dysfunction comprising the steps of: a) identifying an individual suffering from erectile dysfunction; b) selecting a cell with regenerative potential; c) treating said cell with regenerative potential with agents or methods capable of augmenting said regenerative potential as applies to reparation of biological dysfunctions associated with erectile dysfunction; and, d) administering said cell with regenerative potential to said individual suffering from createrectile function.

Further embodiments include a regenerative cell population that comprises stem cells selected from the group consisting of a)embryonic stem cells; b) hematopoietic stem cells; c) mesenchymal stem cells; d) very small embryonic like stem cells; e) inducible pluripotent stem cells; f) bone marrow stem cells; g) amniotic fluid stem cells; h) neuronal stem cells; i) parthenogenically derived stem cells; j) umbilical cord blood stem cells; k) placental stem cells; l) germinal stem cells; m) hair follicle stem cells; n) adipose derived stem cells; o) reprogrammed stem cells; p) peripheral blood derived stem cells; q) peripheral blood mesenchymal stem cells; r) endometrial regenerative cells; s) fallopian tube derived stem cells; and, t) dermal stem cells.

The cell population can include mesenchymal stem cells that are derived from a source selected from the group consisting of: a) bone marrow; b) adipose tissue; c) umbilical cord blood; d) Wharton's Jelly; e) inducible pluripotent generated cells; g) placental tissue; h) peripheral blood mononuclear cells; i) differentiated embryonic stem cells; and, j) differentiated progenitor cells.

Further embodiments are directed to reparations of biological dysfunctions that are selected from the group consisting of: a) Restoration of penile smooth muscle mass and/or function; b) Restoration of penile neural tissue and/or its function; and, c) Restoration of penile blood vessels and/or their function(s).

Further embodiments include regenerative cells that are pre-treated with agents capable of inducing stem cell expansion can be selected from TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, and a member of the bone morphogenic protein family.

Further embodiments include methods capable of augmenting said regenerative potential, selected from the group consisting of: a) Electrical stimulation; b) Shockwave therapy; and, c) Low-level laser irradiation.

Further embodiments include said regenerative cells that are administered intracavernosally to said individual in need thereof.

Further embodiments include methods of treating erectile dysfunction comprising the steps of: a) selecting a cell with regenerative potential; b) administering said cell with regenerative potential to an individual in need of improved erectile function; and, c) administering a therapy to said patient selected from the group consisting of: a) electroacupuncture; b) low-level laser irradiation; and/or, c) shockwave therapy.

Further embodiments include regenerative cell populations that comprise stem cells selected from the group consisting of: a) embryonic stem cells; b) hematopoietic stem cells; c) mesenchymal stem cells; d) very small embryonic like stem cells; e) inducible pluripotent stem cells; f) bone marrow stem cells; g) amniotic fluid stem cells; h) neuronal stem cells; i) parthenogenically derived stem cells; j) umbilical cord blood stem cells; k) placental stem cells; l) germinal stem cells; m) hair follicle stem cells; n) adipose derived stem cells; o) reprogrammed stem cells; p) peripheral blood derived stem cells; q) peripheral blood mesenchymal stem cells; r) endometrial regenerative cells; s) fallopian tube derived stem cells; and, t) dermal stem cells.

Further embodiments include mesenchymal stem cells that are derived from a source selected from the group consisting of: a) bone marrow; b) adipose tissue; c) umbilical cord blood; d) Wharton's Jelly; e) inducible pluripotent generated cells; g) placental tissue; h) peripheral blood mononuclear cells; i) differentiated embryonic stem cells; and, j) differentiated progenitor cells.

Further embodiments include ther term regenerative potential referring to the ability of said regenerative cell to repair biological dysfunctions selected from the group consisting of: a) Restoration of penile smooth muscle mass and/or function; b) Restoration of penile neural tissue and/or its function; and, c) Restoration of penile blood vessels and/or their function(s).

Further embodiments include regenerative cells that are treated with agents capable of inducing stem cell expansion can be selected from TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, and a member of the bone morphogenic protein family.

Further embodiments include regenerative cells that are administered intracavernosally to said individual in need thereof.

### DETAILED DESCRIPTION OF THE EMBODIMENTS

### Definitions

In the context of the present invention, the term “regenerative cell” refers to a mammalian stem cell, progenitor cell, or differentiated cell. As used herein, embryonic, fetal/placental and adult-derived cellular populations are included in the definition of “regenerative cells”. In the context of the present invention, regenerative cells may include: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated tooth derived stem cells, hair follicle stem cells, dermal stem cells, parthenogenically derived stem cells, reprogrammed stem cells and side population stem cells and the like.

As used herein, “stem cell” refers to a pluripotent cell capable of differentiating into numerous cellular lineages.

As used herein, the term “progenitor cell” refers to a lineage-committed cell that may have undergone various stages of differentiation toward a tissue-restricted cell type. The term “differentiated cell” used in the context of the present invention refers to a tissue-restricted cell.

As used herein, the term “mesenchymal stem cell” refers to a multipotent stem cell that can differentiate into a variety of cell types, including: osteoblasts (bone cells), chondrocytes (cartilage cells), and adipocytes (fat cells).

As used herein, “therapeutic energy treatment” refers to any light-, acoustic-, electrical-, or mechanical means of stimulating cells or tissue through means either applied externally to cells (i.e. regenerative cells) or directly to the tissue (specifically, the penis in the context of the present invention) for therapeutic purposes.

As used herein, “low level laser irradiation” refers to photobiomodulation or phototherapy that may be either stimulatory or inhibitory to cellular functions.

As used herein, “biological dysfunctions” used in the context of erectile dysfunction refers to the deficiencies or abnormalities in cellular numbers or functions that underlie the inability of an individual to attain an erection. Said biological dysfunctions are diagnosed in the art using established medical tests and diagnostic criteria. These include but are not limited to the following tests: Doppler Ultrasound, dynamic infusion cavernosometry & cevernosography, tests of penile nerve function including but not limited to the bulbocavernosus reflex test, nocturnal penile tumescence testing, penile biothesiometry, and corpus cavernosometry. Examples of biological dysfunctions present in ED include arteriogenic changes in blood vessels and loss of smooth muscle mass.

As used herein, a “therapeutically effective amount” and “therapeutically sufficient amount” and like terms refer to an amount of an agent sufficient to ameliorate at least one symptom, behavior or event, associated with a pathological, abnormal or otherwise undesirable condition, e.g., erectile dysfunction, or an amount sufficient to prevent or lessen the probability that such a condition will occur or re-occur, or an amount sufficient to delay worsening of such a condition. In one embodiment, the term therapeutically effective amount and like terms are used to refer to the frequencies and concentrations at which regenerative cells are administered for treating erectile dysfunction.

The present invention comprises a combination therapy involving regenerative cells that and therapeutic energy treatments that are administered to a mammal in need of treatment for erectile dysfunction. Said therapeutic energy treatments include electrical stimulation/electroacupuncture, low level laser therapy, and shock wave therapy. Said therapeutic energy treatments can be administered to said regenerative cells in vitro, and said regenerative cells are subsequently administered to an individual in need of treatment for ED. Said therapeutic energy treatments may also be administered directly to a person in need of treatment for ED. In preferred embodiments of the present invention, said therapeutic energy treatments are administered directly into the penis of an individual with ED prior to or subsequent to administration of said regenerative cells to said individual in need thereof. In the context of the present invention, said regenerative cells are utilized as means for regenerating or repairing nerves, blood vessels and/or smooth muscle cells in order to correct biological dysfunctions associated with ED.

In certain aspects, the regenerative cells can be selected either alone or in combination from a group that includes: stem cells, committed progenitor cells, and differentiated cells. The stem cells can be selected from a group that includes: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells and the like.

In one aspect of the invention, an individual in need of treatment for erectile dysfunction is administered one or several doses of the abovementioned cell types at a therapeutically sufficient concentration and/or frequency.

In certain aspects of the present invention, the embryonic stem cells can be totipotent, and can express one or more antigens selected from a group that includes: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT) and the like.

In certain aspects of the present invention, the cord blood stem cells can be multipotent and capable of differentiating into endothelial, smooth muscle, and neuronal cells. The cord blood stem cells can be identified based on expression of one or more antigens selected from a group that includes: SSEA-3, SSEA-4, CD9, CD34, c-kit, OCT-4, Nanog, and CXCR-4 and the like. Further, the cord blood stem cells selected may not express one or more markers selected from a group that includes: CD3, CD34, CD45, and CD11b and the like.

In certain aspects of the present invention, the placental stem cells can be isolated from the placental structure, and can be identified based on expression of one or more antigens selected from a group that includes: Oct-4, Rex-1, CD9, CD13, CD29, CD44, CD166, CD90, CD105, SH-3, SH-4, TRA-1-60, TRA-1-81, SSEA-4 and Sox-2 and the like.

In certain aspects of the present invention, the bone marrow stem cells can be bone marrow mononuclear cells, and can be selected based on the ability to differentiate into one or more of the following cell types: endothelial cells, smooth muscle cells, and neuronal cells. The bone marrow stem cells can be selected based on expression of one or more of the following antigens: CD34, c-kit, flk-1, Stro-1, CD105, CD73, CD31, CD146, vascular endothelial-cadherin, CD133 and CXCR-4. Further, the bone marrow stem cells can be enriched for expression of CD133.

In certain aspects of the present invention, the amniotic fluid stem cells can be isolated by introduction of a fluid extraction means into the amniotic cavity under ultrasound guidance. The amniotic fluid stem cells can be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1. Further, the amniotic fluid stem cells can be selected based on lack of expression of one or more of the following antigens: CD34, CD45, and HLA Class II.

In certain aspects of the present invention, the neuronal stem cells can be selected based on expression of one or more of the following antigens: RC-2, 3CB2, BLB, Sox-2hh, GLAST, Pax 6, nestin, Muashi-1, NCAM, A2B5 and prominin.

In certain aspects of the present invention, the circulating peripheral blood stem cells can be characterized by ability to proliferate in vitro for a period of over 3 months. Further, the circulating peripheral blood stem cells can be characterized by expression of CD34, CXCR4, CD117, CD113, and c-met. Further, the circulating peripheral blood stem cells may lack substantial expression of differentiation associated markers, such as, for example CD2, CD3, CD4, CD11, CD11a, Mac-1, CD14, CD16, CD19, CD24, CD33, CD36, CD38, CD45, CD56, CD64, CD68, CD86, CD66b, and HLA-DR and the like.

In certain aspects of the present invention, the mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Further, the mesenchymal stem cells may not express substantial levels of HLA-DR, CD117, and CD45. In certain aspects, the mesenchymal stem cells can be derived from a group selected of: bone marrow, adipose tissue, umbilical cord blood, placental tissue, peripheral blood mononuclear cells, differentiated embryonic stem cells, and differentiated progenitor cells.

In certain aspects of the present invention, the germinal stem cells express markers selected from a group that includes: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1 and the like.

In certain aspects of the present invention, the adipose tissue derived stem cells express markers selected from a group that includes: CD13, CD29, CD44, CD63, CD73, CD90, CD166, Aldehyde dehydrogenase (ALDH), and ABCG2 and the like. The adipose tissue derived stem cells can be a population of purified mononuclear cells extracted from adipose tissue capable of proliferating in culture for more than 1 month.

In certain aspects of the present invention, the exfoliated teeth derived stem cells express markers selected from a group that includes: STRO-1, CD146 (MUC18), alkaline phosphatase, MEPE, and bFGF and the like.

In certain aspects of the present invention, the hair follicle stem cells express markers selected from a group that includes: cytokeratin 15, Nanog, and Oct-4 and the like, and can be capable of proliferating in culture for a period of at least one month. The hair follicle stem cells may secrete one or more of the following proteins when grown in culture: basic fibroblast growth factor (bFGF), endothelin-1 (ET-1) and stem cell factor (SCF).

In certain aspects of the present invention, the dermal stem cells express markers selected from a group that includes: CD44, CD13, CD29, CD90, and CD105 and the like, and can be capable of proliferating in culture for a period of at least one month.

In certain aspects of the present invention, the parthenogenically derived stem cells can be generated by addition of a calcium flux inducing agent to activate an oocyte followed by enrichment of cells expressing markers selected from a group that includes SSEA-4, TRA 1-60 and TRA 1-81 and the like.

In certain aspects of the present invention, the progenitor cells can be selected from a group that includes: endothelial progenitor cells, neuronal progenitor cells, and hematopoietic progenitor cells and the like.

In certain aspects, the committed endothelial progenitor cells express markers selected from a group that includes: CD31, CD34, AC133, CD146 and flk1 and the like.

In certain aspects of the present invention, the committed hematopoietic cells can be purified from the bone marrow, or from peripheral blood, such as from peripheral blood of a patient whose committed hematopoietic progenitor cells can be mobilized by administration of a mobilizing agent or therapy. The mobilizing agent can be selected from a group that includes: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA-reductase inhibitors and small molecule antagonists of SDF-1 and the like. Further, the mobilization therapy can be selected from a group that includes: exercise, hyperbaric oxygen, autohemotherapy by ex vivo ozonation of peripheral blood, and induction of SDF-1 secretion outside of the bone marrow.

In certain aspects of the present invention, the committed hematopoietic progenitor cells can express the marker CD133 or CD34.

In specific embodiments of the invention, the regenerative cells are expanded in culture using one or more cytokines, chemokines and/or growth factors prior to administration to an individual in need thereof. The agent capable of inducing stem cell expansion can be selected from TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, and a member of the bone morphogenic protein family. The agent capable of inducing stem cell differentiation can be selected from HGF, BDNF, VEGF, FGF1, FGF2, FGF4, and FGF 20.

For use in the present invention, said regenerative cells may be administered to an individual in need thereof via one or more of the following routes of administration: intravenously, intramuscularly, intraperitoneally, transdermally, or by any parenteral route. In a preferred embodiment of the present invention, said regenerative cells are administered intracavernosally.

Furthermore, conditions promoting certain type of cellular proliferation or differentiation can be used during the culture of said regenerative cells. These conditions include but are not limited to, alteration in temperature, alternation in oxygen/carbon dioxide content, alternations in turbidity of said media, or exposure to small molecules modifiers of cell cultures such as nutrients, inhibitors of certain enzymes, stimulators of certain enzymes, inhibitors of histone deacetylase activity such as valproic acid (Bug, et al., 2005, Cancer Res 65:2537-2541), trichostatin-A (Young, et al., 2004, Cytotherapy 6:328-336), trapoxin A (Kijima, et al., 1993, J Biol Chem 268:22429-22435), or Depsipeptide (Gagnon, et al., 2003, Anticancer Drugs 14:193-202; Fujieda, et al., 2005, Int J Oncol 27:743-748), inhibitors of DNA methyltransferase activity such as 5-azacytidine, inhibitors of the enzyme GSK-3 (Trowbridge, et al., 2006, Nat Med 12:89-98, and the like.

For use in the present invention, said regenerative cells may be administered to an individual in need thereof via one or more of the following routes of administration: intravenously, intramuscularly, intraperitoneally, transdermally, or by any parenteral route. In a preferred embodiment of the present invention, said regenerative cells are administered intracavernosally (i.e. directly into the corpus cavernosa of the penis).

In a preferred embodiment of the present invention, said regenerative cells are treated with a therapeutic energy treatment prior to administration to an individual afflicted with erectile dysfunction. Said therapeutic energy treatment may be administered to regenerative cells in vitro and can be selected from one or more of the following: electrical stimulation of cells, low-level laser irradiation of cells, and/or shockwave therapy of cells. Without being bound by theory, these therapeutic energy treatments can increase the therapeutic efficacy of the cellular product used to treat erectile dysfunction by altering one or more of the following activities of regenerative cells: proliferation, differentiation, survival, cytokine and chemokine production, and migration/homing to specific tissue sites.

In one embodiment, the current invention is practiced by pre-treating regenerative cells with electrical stimulation to modify their therapeutic potential for treating ED. Methods for electrically stimulating cells ex vivo are known in the art. One method for practicing the disclosed invention is described in [38]. Briefly, regenerative cells can be subjected to electrical stimulation using an electrical stimulator consisting of 16 gold electrodes coated with platinum that fit into 8-well chamber slides. Each well contains an anode and cathode with an interelectrode distance of 10 mm. The electrodes can be connected to an electrical stimulator that generates charge-balanced biphasic current pulses. The cells can then be electrically stimulated in suspension for different durations (for example, between 5 and 15 minutes with electric fields of 200 mV/mm at 1 Hz frequencies and 1 ms pulse width). Control regenerative cells can be subjected to the same procedure but without electrical stimulation. After electrical stimulation, the regenerative cells can be cultured in media prior to administration to an individual in need thereof.

Other devices/apparatus for delivering said electrical stimulation are applicable to the present invention. In the current invention, a therapeutically effective amount of electrical stimulation can be delivered for a period of time sufficient to evoke desirable gene- and protein-expression patterns in said regenerative cells including but not limited to the following: a) Expression of angiogenesis-related genes or proteins; b) Expression of genes or proteins associated with smooth muscle cells or their precursors; and/or, c) Expression of genes or proteins associated with nerve cells or their precursors. Using techniques established in the art to identify one or more of the aforementioned categories of genes and/or proteins, the regenerative potential of electrically-stimulated cells can be compared to that of control cells (non-electrically stimulated) and the suitability of the former for therapeutic purposes (i.e. for treated an individual with ED) can be screened.

In another embodiment of this invention, regenerative cells can be treated with low-level laser irradiation in vitro to alter their therapeutic potential for the treatment of ED. Techniques for delivering low-level laser irradiation are known in the art. In one embodiment, Low reactive level laser therapy (LLLT) will be delivered directly to cells in tissue culture plates using a laser devices that are commercially available. This invention may also be practiced using different types of laser therapies having variable wavelengths, power outputs/densities, energy densities, types of exposure, and treatment durations. Examples of different types of therapies include single, pulsed, super-pulsed exposure and continuous mode. Various lasers can be utilized including but not limited to diode lasers, He—Ne lasers, Er:YAG (Erbium-Doped Yttrium Aluminum Garnet) lasers, and Superpulsed low-level laser therapy (SLLLT). A therapeutically effective amount of laser stimulation can be delivered for a period of time sufficient to evoke desirable gene- and protein-expression patterns in said regenerative cells including but not limited to the following: a) Expression of angiogenesis-related genes or proteins; b) Expression of genes or proteins associated with smooth muscle cells or their precursors; and/or, c) Expression of genes or proteins associated with nerve cells or their precursors.

In another embodiment of this invention, regenerative cells can be treated with electrocorporeal shockwave therapy in vitro to alter their therapeutic potential for the treatment of ED. This invention may be practiced using methods similar to those described in [39]. Briefly, shockwaves can be applied with a defocused Dermagold 100 device and an OP155 applicator (MTS Medical, Konstanz, Germany). The cells can be stimulated in T25 cell culture flasks, in 15 ml or in 50 ml tubes in PBS. Cells will be submerged in a water bath and stimulated with a frequency of 5 Hz, 200 pulses and energy flux densities ranging from 0.03 to 0.19 mJ/mm² at a constant pressure level of 1 bar. A therapeutically effective amount of extracorporeal shockwave therapy can be delivered for a period of time sufficient to evoke desirable gene- and protein-expression patterns in said regenerative cells including but not limited to the following: a) Expression of angiogenesis-related genes or proteins; b) Expression of genes or proteins associated with smooth muscle cells or their precursors; and/or, c) Expression of genes or proteins associated with nerve cells or their precursors.

Also provided is a method of treating erectile dysfunction comprising administering a therapeutically effective amount of regenerative cells capable of inducing one or more biological activities selected from the group that includes: a) inducing regeneration of nervous tissue; b) stimulating smooth muscle cell activity; c) stimulating increased perfusion or angiogenesis; and, also co-administering therapeutic energy treatments to said individual in need thereof. Said therapeutic energy treatments are selected from one or more of the following: electroacupuncture or TENS, low-level laser irradiation, and/or extracorporeal shockwave therapy. Said therapeutic energy treatments can be administered directly to the penis. Electroacupuncture can be administered at a site distant from the penis using techniques known in the art of alternative medicine. While not being bound by theory, said therapeutic energy treatments can be utilized to enhance the regenerative activities of said regenerative cells or to improve their migration to the penis and/or their retention in the penis. In a preferred embodiment, said regenerative cells may be injected directly into the corpus cavernosa and low-level laser irradiation or extracorporeal shockwave therapy are also administered directly to the penis. Alternatively, said regenerative cells may be injected via another route of administration (for example, intravenously) and said therapeutic energy treatments can be administered to the penile tissue to facilitate migration, survival or other activities of regenerative cells in the penis.

Thus, provided herein is a method of treating or preventing the onset of erectile dysfunction in a mammal comprising administering a therapeutically effective amount of cells capable of inducing one or more biological activities selected from the group that includes: a) inhibiting neuronal cell dysfunction, b) inhibiting cavernosal fibrosis, c) inhibiting smooth muscle degeneration, and d) inhibiting biological pathways causative of ischemia and the like. Said biological activities of regenerative cells can be modulated by direct treatment of cells with electrical stimulation, shockwave therapy, or low-level laser irradiation. Alternatively, regenerative cells can be administered to an individual for treating erectile dysfunction either as a monotherapy or in a combination therapy with electroacupuncture, low-level laser irradiation, and/or shockwave therapy administered directly to the subject.

Also provided are methods of treating or preventing the onset of erectile dysfunction in a mammal comprising administering a therapeutically effective amount of cells capable of inducing one or more biological activities selected from the group that includes: a) inducing regeneration of nervous tissue; b) stimulating smooth muscle cell activity; c) stimulating increased perfusion or angiogenesis and the like.

### REFERENCES

1. Feldman, H. A., et al., Impotence and its medical and psychosocial correlates: results of the Massachusetts Male Aging Study. J Urol, 1994. 151(1): p. 54-61.

2. Clavijo, R. I., M. M. Miner, and J. Rajfer, Erectile Dysfunction and Essential Hypertension: The Same Aging-related Disorder? Rev Urol, 2014. 16(4): p. 167-71.

3. Magheli, A. and A. L. Burnett, Erectile dysfunction following prostatectomy: prevention and treatment. Nat Rev Urol, 2009. 6(8): p. 415-27.

4. Tal, R., et al., Persistent erectile dysfunction following radical prostatectomy: the association between nerve-sparing status and the prevalence and chronology of venous leak. J Sex Med, 2009. 6(10): p. 2813-9.

5. Nolan, M. W., et al., Pudendal nerve and internal pudendal artery damage may contribute to radiation-induced erectile dysfunction. Int J Radiat Oncol Biol Phys, 2015. 91(4): p. 796-806.

6. Moore, C. S., et al., Erectile dysfunction, vascular risk, and cognitive performance in late middle age. Psychol Aging, 2014. 29(1): p. 163-72.

7. Chaudhary, R. K., et al., Risk factors for erectile dysfunction in patients with cardiovascular disease. J Int Med Res, 2016.

8. Binmoammar, T. A., et al., The impact of poor glycaemic control on the prevalence of erectile dysfunction in men with type 2 diabetes mellitus: a systematic review. JRSM Open, 2016. 7(3): p. 2054270415622602.

9. Bleustein, C. B., et al., The neuropathy of erectile dysfunction. Int J Impot Res, 2002. 14(6): p. 433-9.

10. Hurt, K. J., et al., Cyclic AMP-dependent phosphorylation of neuronal nitric oxide synthase mediates penile erection. Proc Natl Acad Sci USA, 2012. 109(41): p. 16624-9.

11. Ferguson, J. E., 3rd and C. C. Carson, 3rd, Phosphodiesterase type 5 inhibitors as a treatment for erectile dysfunction: Current information and new horizons. Arab J Urol, 2013. 11(3): p. 222-9.

12. Rendell, M. S., et al., Sildenafil for treatment of erectile dysfunction in men with diabetes: a randomized controlled trial. Sildenafil Diabetes Study Group. JAMA, 1999. 281(5): p. 421-6.

13. Martinez-Jabaloyas, J. M., et al., Prognostic factors for response to sildenafil in patients with erectile dysfunction. Eur Urol, 2001. 40(6): p. 641-6; discussion 647.

14. Deng, W., et al., Gene and stem cell therapy for erectile dysfunction. Int J Impot Res, 2005. 17 Suppl 1: p. S57-63.

15. Okolicsanyi, R. K., et al., Human Mesenchymal Stem Cells Retain Multilineage Differentiation Capacity Including Neural Marker Expression after Extended In Vitro Expansion. PLoS One, 2015. 10(9): p. e0137255.

16. Foudah, D., et al., Expression of neural markers by undifferentiated mesenchymal-like stem cells from different sources. J Immunol Res, 2014. 2014: p. 987678.

17. Eming, S. A., T. Krieg, and J. M. Davidson, Inflammation in wound repair: molecular and cellular mechanisms. J Invest Dermatol, 2007. 127(3): p. 514-25.

18. Luster, A. D., R. Alon, and U. H. von Andrian, Immune cell migration in inflammation: present and future therapeutic targets. Nat Immunol, 2005. 6(12): p. 1182-90.

19. Ding, Y., et al., Electro-acupuncture promotes survival, differentiation of the bone marrow mesenchymal stem cells as well as functional recovery in the spinal cord-transected rats. BMC Neurosci, 2009. 10: p. 35.

20. Genovese, J. A., et al., Electrostimulation induces cardiomyocyte predifferentiation of fibroblasts. Biochem Biophys Res Commun, 2008. 370(3): p. 450-5.

21. Genovese, J. A., et al., Cardiac pre-differentiation of human mesenchymal stem cells by electrostimulation. Front Biosci (Landmark Ed), 2009. 14: p. 2996-3002.

22. Pietronave, S., et al., Monophasic and biphasic electrical stimulation induces a precardiac differentiation in progenitor cells isolated from human heart. Stem Cells Dev, 2014. 23(8): p. 888-98.

23. Pavesi, A., et al., Electrical conditioning of adipose-derived stem cells in a multi-chamber culture platform. Biotechnol Bioeng, 2014. 111(7): p. 1452-63.

24. Kushibiki, T., et al., Low Reactive Level Laser Therapy for Mesenchymal Stromal Cells Therapies. Stem Cells Int, 2015. 2015: p. 974864.

25. Stein, A., et al., Low-level laser irradiation promotes proliferation and differentiation of human osteoblasts in vitro. Photomed Laser Surg, 2005. 23(2): p. 161-6.

26. Stein, E., et al., Initial effects of low-level laser therapy on growth and differentiation of human osteoblast-like cells. Wien Klin Wochenschr, 2008. 120(3-4): p. 112-7.

27. Barboza, C. A., et al., Low-level laser irradiation induces in vitro proliferation of mesenchymal stem cells. Einstein (Sao Paulo), 2014. 12(1): p. 75-81.

28. Yu, W., J. O. Naim, and R. J. Lanzafame, The effect of laser irradiation on the release of bFGF from 3T3 fibroblasts. Photochem Photobiol, 1994. 59(2): p. 167-70.

29. Shen, C. C., et al., Low-level laser stimulation on adipose-tissue-derived stem cell treatments for focal cerebral ischemia in rats. Evid Based Complement Alternat Med, 2013. 2013: p. 594906.

30. Waldinger, M. D., et al., Penile anesthesia in Post SSRI Sexual Dysfunction (PSSD) responds to low-power laser irradiation: a case study and hypothesis about the role of transient receptor potential (TRP) ion channels. Eur J Pharmacol, 2015. 753: p. 263-8.

31. Wu, J. Y., et al., Low-power laser irradiation suppresses inflammatory response of human adipose-derived stem cells by modulating intracellular cyclic AMP level and NF-kappaB activity. PLoS One, 2013. 8(1): p. e54067.

32. Wu, S., et al., High fluence low-power laser irradiation induces mitochondrial permeability transition mediated by reactive oxygen species. J Cell Physiol, 2009. 218(3): p. 603-11.

33. Zhou, F., et al., The TGF-beta1/Smad/CTGF pathway and corpus cavernosum fibrous-muscular alterations in rats with streptozotocin-induced diabetes. J Androl, 2012. 33(4): p. 651-9.

34. Liu, J., et al., Evaluation of the effect of different doses of low energy shock wave therapy on the erectile function of streptozotocin (STZ)-induced diabetic rats. Int J Mol Sci, 2013. 14(5): p. 10661-73.

35. Olsen, A. B., et al., Can low-intensity extracorporeal shockwave therapy improve erectile dysfunction? A prospective, randomized, double-blind, placebo-controlled study. Scand J Urol, 2015. 49(4): p. 329-33.

36. Vardi, Y., et al., Can low-intensity extracorporeal shockwave therapy improve erectile function? A 6-month follow-up pilot study in patients with organic erectile dysfunction. Eur Urol, 2010. 58(2): p. 243-8.

37. Gruenwald, I., et al., Shockwave treatment of erectile dysfunction. Ther Adv Urol, 2013. 5(2): p. 95-9.

38. Hernandez, D., et al., Electrical Stimulation Promotes Cardiac Differentiation of Human Induced Pluripotent Stem Cells. Stem Cells Int, 2016. 2016: p. 1718041.

39. Rohringer, S., et al., Molecular and cellular effects of in vitro shockwave treatment on lymphatic endothelial cells. PLoS One, 2014. 9(12): p. e114806.

1. A method of treating erectile dysfunction comprising the steps of: a) identifying an individual suffering from erectile dysfunction; b) selecting a cell with regenerative potential; c) treating said cell with regenerative potential with agents or methods capable of augmenting said regenerative potential as applies to reparation of biological dysfunctions associated with erectile dysfunction; and, d) administering said cell with regenerative potential to said individual suffering from createrectile function.

2. The method of claim 1, wherein said regenerative cell population comprises stem cells selected from the group consisting of a)embryonic stem cells; b) hematopoietic stem cells; c) mesenchymal stem cells; d) very small embryonic like stem cells; e) inducible pluripotent stem cells; f) bone marrow stem cells; g) amniotic fluid stem cells; h) neuronal stem cells; i) parthenogenically derived stem cells; j) umbilical cord blood stem cells; k) placental stem cells; l) germinal stem cells; m) hair follicle stem cells; n) adipose derived stem cells; o) reprogrammed stem cells; p) peripheral blood derived stem cells; q) peripheral blood mesenchymal stem cells; r) endometrial regenerative cells; s) fallopian tube derived stem cells; and, t) dermal stem cells.

3. The cell population of claim 2, wherein said mesenchymal stem cells are derived from a source selected from the group consisting of: a) bone marrow; b) adipose tissue; c) umbilical cord blood; d) Wharton's Jelly; e) inducible pluripotent generated cells; g) placental tissue; h) peripheral blood mononuclear cells; i) differentiated embryonic stem cells; and, j) differentiated progenitor cells.

4. The method of claim 1, wherein said reparation of biological dysfunctions is selected from the group consisting of: a) Restoration of penile smooth muscle mass and/or function; b) Restoration of penile neural tissue and/or its function; and, c) Restoration of penile blood vessels and/or their function(s).

5. The method of claim 1, wherein said regenerative cells are pre-treated with agents capable of inducing stem cell expansion can be selected from TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, and a member of the bone morphogenic protein family.

6. The method of claim 1, wherein said methods capable of augmenting said regenerative potential are selecting from the group consisting of: a) Electrical stimulation; b) Shockwave therapy; and, c) Low-level laser irradiation.

7. The method of claim 1, wherein said regenerative cells are administered intracavernosally to said individual in need thereof.

8. A method of treating erectile dysfunction comprising the steps of: a) selecting a cell with regenerative potential; b) administering said cell with regenerative potential to an individual in need of improved erectile function; and, c) administering a therapy to said patient selected from the group consisting of: a) electroacupuncture; b) low-level laser irradiation; and/or, c) shockwave therapy.

9. The method of claim 1, wherein said regenerative cell population comprises stem cells selected from the group consisting of: a) embryonic stem cells; b) hematopoietic stem cells; c) mesenchymal stem cells; d) very small embryonic like stem cells; e) inducible pluripotent stem cells; f) bone marrow stem cells; g) amniotic fluid stem cells; h) neuronal stem cells; i) parthenogenically derived stem cells; j) umbilical cord blood stem cells; k) placental stem cells; l) germinal stem cells; m) hair follicle stem cells; n) adipose derived stem cells; o) reprogrammed stem cells; p) peripheral blood derived stem cells; q) peripheral blood mesenchymal stem cells; r) endometrial regenerative cells; s) fallopian tube derived stem cells; and, t) dermal stem cells.

10. The cell population of claim 2, wherein said mesenchymal stem cells are derived from a source selected from the group consisting of: a) bone marrow; b) adipose tissue; c) umbilical cord blood; d) Wharton's Jelly; e) inducible pluripotent generated cells; g) placental tissue; h) peripheral blood mononuclear cells; i) differentiated embryonic stem cells; and, j) differentiated progenitor cells.

11. The method of claim 1, wherein said regenerative potential refers to the ability of said regenerative cell to repair biological dysfunctions selected from the group consisting of: a) Restoration of penile smooth muscle mass and/or function; b) Restoration of penile neural tissue and/or its function; and, c) Restoration of penile blood vessels and/or their function(s).

12. The method of claim 1, wherein said regenerative cells are treated with agents capable of inducing stem cell expansion can be selected from TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, and a member of the bone morphogenic protein family.

13. The method of claim 1, wherein said regenerative cells are administered intracavernosally to said individual in need thereof.
